(19) United States
(12) Patent Application Publication
(10) Pub. No.: US 2018/0160970 A1
(43) Pub. Date: Jun. 14, 2018

(54) DEVICE FOR DIAGNOSING THE EFFICACY OF VENTILATION OF A PATIENT AND METHOD FOR DETERMINING THE VENTILATORY EFFICACY OF A PATIENT

(71) Applicants: POLYCAPTIL, Besancon (FR); UNIVERSITE DE FRANCHE-COMTE, Besancon (FR); CENTRE HOSPITALIER REGIONAL UNIVERSITAIRE DE BESANCON, Besancon (FR)

(72) Inventors: Abdo KHOURY, Besancon (FR); Alban DE LUCA, Besancon (FR); Fatimata Seydou SALL, Besancon (FR); Lionel PAZART, Besancon (FR); Gilles CAPELLIER, Grandfontaine (FR); Pierre-Edouard SAILLARD, Besancon (FR); Florin Dan NITA, Serre-Les-Sapins (FR); Jean-Francois VINCHANT, Besancon (FR)

(73) Assignees: POLYCAPTIL, Besancon (FR); UNIVERSITE DE FRANCHE-COM, Besancon (FR); CENTRE HOSPITALIER REGIONAL UNIVERSITAIRE DE BESANCON, Besancon (FR)

(21) Appl. No.: 15/580,526

(22) PCT Filed: May 30, 2016
(86) PCT No.: PCT/EP2016/062162

§ 371 Date: Dec. 7, 2017

(30) Foreign Application Priority Data
Jun. 8, 2015 (FR) 1555220

(51) Int. Cl. A61B 5/00; A61B 5/087; A61M 16/00

(52) U.S. Cl. CPC A61B 5/4848; A61B 5/0878; A61B 5/4836; A61M 16/0051; A61M 16/0078; A61M 16/0084; A61M 2230/432; A61M 2016/0027; A61M 2205/15; A61M 2205/18; A61M 2205/273; A61M 2205/502; A61M 2205/584; A61M 16/024

(57) ABSTRACT

A device for diagnosing the ventilatory efficacy of a patient under respiratory assistance, said device being intended to cooperate with a system for ventilating the patient, the device having: a bidirectional thermal mass sensor for measuring, in real time, the air flows during insufflation and during exhalation, an electronic casing connected to said sensor and configured to receive and process data relating to the air flows measured by the sensor, the electronic casing having: i. a user interface comprising a display device and data input means, ii. a data-processing center, the data-processing center functioning according to programmed algorithms for acquiring, processing and displaying the data, for analyzing the efficacy of the ventilation in real time, and for managing alarms, and iii. means for supplying electricity.

The present invention relates to a device for diagnosing the ventilatory effectiveness of a patient under respiratory assistance. The invention also relates to a ventilation system for providing respiratory assistance to a patient including such a device and to a method for determining the ventilatory effectiveness of a patient using such a ventilation system.

Ventilation systems are used by first responders and medical personnel and paramedics responding to emergencies, in anesthesia and reanimation inside or outside a hospital or other health center.

A plurality of research projects have been undertaken and a plurality of devices have been developed over the last few years to improve the effectiveness of manual ventilation.

US2013/0180527 relates to the optimization of the shape of the bag, used for the ventilation, including spots for fingers in order to make sure one and only one compression method is used, thus decreasing the variation in the volume of air delivered to the patient. This apparatus was designed to deliver a constant volume between 500 and 600 ml.

US2008/0236585 measures the air flow rates and the peak pressure at the insufflation valve, indicates to the first responders the ideal frequencies of ventilation by way of a luminous rate signal and displays the volume insufflated in each ventilatory cycle.

WO2014/078840 describes a system and a method for controlling the reanimation and the respiratory function of a patient. A pressure sensor detects the pressure of the air and generates a first detection signal. A flow rate sensor measures an air flow rate and generates a second signal. A processor receives and processes the first and second detection signals using an algorithm to identify a ventilatory frequency, a pulmonary pressure and a volume of air delivered to the patient. An analysis report is generated in real-time with these identified values.

Among commercially available devices, Medumat Easy CPR from the company Weinmann is a less expensive alternative to mechanical transportable ventilators. This device delivers a manually triggered artificial ventilation at positive pressure and has a rate function allowing the first responder to respect the optimal ventilation frequency such as described in international emergency medicine recommendations. This device has been evaluated in a few studies and a priori decreases the dispersion in ventilatory parameters such as ventilation frequency and insufflated volumes. However, its use requires a source of pressurized oxygen. In addition, the first responder is required to be knowledgeable in respiratory physiology and management of respiratory tracts in order to be able to adjust the ventilatory parameters depending on the clinical state of the patient.

Another known device sold commercially under the trademark Exhalometer™ by the company Galemed Corporation is intended to measure tidal volume, minute volume and the ventilation frequency delivered to the patient. This device measures the amount of air passing through the expiratory valve of the bag, which may differ significantly from the actual tidal volume. Specifically, many studies have shown a high quantity of leaks, i.e. between 25 and 40% leaks, during mask ventilation, so that the expired volume passing through the Exhalometer™ is decreased by leaks that occur during the insufflation and the expiration between the mask and the face of the patient.

These two devices do not allow the effectiveness of the ventilation taking into account the clinical state of the patient to be evaluated and do not have a function allowing hyperventilation to be decreased or warning messages to be delivered to the first responders.

There is thus a need to provide a ventilation system that takes into account the clinical state of the patient.

There is also a need to provide a ventilation system that is usable by any first responder and medical personnel or paramedic responding in a health center or outside, without in-depth prior training.

Lastly, there is a need to provide a ventilation system that allows rapid correction of poor ventilation.

To meet all or some of the aforementioned needs, the present invention provides a device for diagnosing the ventilatory effectiveness of a patient placed under respiratory assistance, intended to interact with a system for ventilating the patient, the device including:
a two-way thermal mass sensor, able to measure in real-time the air flow rates on insufflation and on expiration;
an electronic unit connected to said sensor, configured to receive and process data relating to the air flow rates measured by the sensor.

By virtue of the presence of the two-way thermal mass sensor, it is possible to measure air flow rates on insufflation and on expiration by way of the measurement of a temperature gradient that is correlated to the amount of gaseous fluid flowing therethrough. This sensor opposes no significant resistance to the air flow, whether this be on inspiration or on expiration, and allows a calibration of the measurement depending on temperature, pressure, and the composition of the fluid (air, O2, N2) and is not sensitive to gravity or to the orientation of the device. Contrary to the pressure-gradient flowmeters used in present-day mechanical ventilators, this technology has the advantage of being both more precise without however opposing resistance to the insufflation or an expiratory obstacle to the patient.

The sensor is preferably single-use. As a variant, the sensor may be autoclavable. The use of such a single-use or autoclavable two-way thermal mass sensor makes it possible to avoid the use of a filter, which is a substantial obstacle to the ventilation and to the measurement of the ventilatory parameters because of its bulk and its resistance to the air flows.

By “respiratory assistance” or “ventilatory assistance”, what is meant is any type of respiratory assistance, whether it be partial or total, total respiratory assistance also being called respiratory replacement.

The diagnostic device may be associated with any device for ventilating a patient, for any type of ventilatory necessity and with any type of invasive or non-invasive interface.

By virtue of the invention, a device for diagnosing ventilatory effectiveness is provided that is compact and light, and placed as close as possible to the patient upstream of the mask or the tube in order to measure the respiratory parameters of the patient. Its compactness in addition allows a smaller casing to be used. Its low weight improves its handleability and its use.

The device preferably includes a disconnectable connection between the sensor and the electronic unit.

The sensor and the electronic unit may be connected easily, without a tool or particular know-how, via an electro-mechanical connection.

As a variant, the link between the sensor and the electronic unit is wireless.

The electronic unit may include:
a user interface comprising a display device such as a screen and means for inputting data;
a data-processing center;
a means for supplying electrical power such as at least one battery.

When one or more batteries are present, there is no need to plug the device into the mains, this allowing the diagnostic device to be used anywhere.

The data-processing center for example operates according to algorithms programmed to acquire, to process and to display data, to analyze the effectiveness of the ventilation in real-time and to manage alarms, in particular such as described below.

The electronic unit may take the form of a microprocessor, connected by an optionally wired link to the two-way thermal mass sensor. The user interface and the data-processing center and any other component of the electronic unit may be located within one and the same apparatus or be separate or remote from each other or one another.

The diagnostic device may even include at least one other sensor, chosen from the following sensors: a pressure sensor, and a sensor of CO₂ concentration in the air. Such sensors may allow pulmonary characteristics and characteristics of the clinical state of the patient to be measured, which will then be analyzed by the data-processing center of the electronic unit of the device. When they are present, the one or more other sensors may be integrated into the diagnostic device. As a variant, they may be present in the ventilation system with which the diagnostic device interacts.

The diagnostic device allows the actual tidal volume to be evaluated, allowing control of and information to be given on the actual amount of air participating in the gas exchange. It performs a tailored analysis in real-time of the ventilatory effectiveness with regard to the physiological characteristics of the patient. The device delivers to the first responder warning and advisory messages in order to make it so that an adequate ventilation is maintained in all circumstances. The diagnostic device takes into account the physiological characteristics of the patient in order to give information to the first responder on the right ventilation frequency, in particular via a luminous and/or audio signal, and to display the actual tidal volume that must be delivered to the patient.

The device for diagnosing the ventilatory effectiveness of the patient under respiratory assistance allows adjustment in real-time of the ventilatory parameters applied to the patient consistent with his recommended needs or the evolution of his clinical state.

By “physiological characteristics of the patient” or “physiological parameters of the patient”, what is meant is any physical quantity that characterizes the intrinsic properties of the patient either on the level of mechanical characteristics of the respiratory system, such as lung capacity, pulmonary compliance, pulmonary resistance, expiratory time constant, inter alia, or of variables resulting from the interaction between the ventilation of the patient and other physiological systems, and in particular the cardiovascular system, such as the concentration of CO₂ in the expired air, arterial oxygen saturation, inter alia.

By “ventilatory parameters”, what is meant is the measured parameters corresponding to the implementation of the respiratory assistance on the patient.

Yet another subject of the invention, according to another of its aspects, in combination with the above, is a ventilation system for providing respiratory assistance to a patient, including a device for diagnosing the effectiveness of the ventilation of a patient such as defined above, and a ventilation device chosen from the group consisting of: a flexible bag, a self-inflating bag and a mechanical ventilator.

The ventilation system is preferably able to be suitable for a use chosen from the group consisting of: continuous ventilation of a patient in respiratory distress, respiratory replacement for an apneic patient, spontaneous ventilation of a patient and discontinuous ventilation of a patient in cardiac arrest.

The ventilation system advantageously includes a ventilation interface chosen from the group consisting of: an invasive ventilation via tracheotomy or tracheal tube, and a non-invasive ventilation via a mask.

Yet another subject of the invention, according to another of its aspects, independently of or in combination with the above, is a method for determining the ventilatory effectiveness of a patient using a ventilation system, in particular such as defined above or any other adequate ventilation system, comprising at least a ventilation device, a ventilation interface and one or more sensors of air flow rate, pressure and/or CO₂ concentration in the air and an associated microprocessor, this method being characterized in that it includes the following steps:
a) allowing physical and/or physiological characteristics of the patient to be input into the electronic unit, and/or characteristics relating to the ventilation, in particular relating to the type of ventilation, to the type of ventilation device and/or to the type of ventilation interface to be input;
b) measuring the physiological parameters of the patient using the one or more sensors;
c) analyzing the characteristics input in step a) and the parameters measured in step b);
d) deducing therefrom, in real-time, ideal ventilatory parameters for an optimal ventilation of said patient, and for each ventilatory parameter, a minimum and/or maximum threshold;
e) measuring in real-time the ventilatory parameters of the patient;
f) comparing the measured ventilatory parameters to said thresholds, respectively;
g) for each ventilatory parameter, in case of value of a measured ventilatory parameter higher than a corresponding maximum threshold and/or lower than a corresponding minimum threshold, generating an alarm and/or a piece of information on the one or more parameters to be modified or corrections to be carried out to achieve an optimal ventilation;
h) repeating steps b) to g) throughout the duration of the ventilatory assistance provided to the patient, in particular in each ventilation cycle.

By virtue of the method according to the invention, in particular in its steps c) and d), it is possible to perform a diagnosis of physiological characteristics of the patient placed under respiratory assistance and to adjust in real-time the ventilatory parameters applied to the patient consistent with his recommended needs or the evolution of his clinical state, and to adjust the alarm thresholds accordingly.

The method consists according to the invention in carrying out a continuous and automatic interpretation of respiratory curves. A system for managing warning messages allows the first responder to be warned in case of dangerous ventilation and the most effective way of recovering an adequate ventilation to be indicated thereto. The objective is to detect the parameter having a negative impact on the ventilatory effectiveness and to display specific messages to the first responder in order to regain a satisfactory level of effectiveness as rapidly and simply as possible. A plurality of problems may arise when the ventilation is insufficient or excessive and the role of this key function is therefore to indicate which of these parameters may be corrected prioritarily in order to ensure an effective ventilation.

The physical and/or physiological characteristics and parameters of the patient advantageously comprise at least two from the following characteristics or parameters: the size of the patient, his lung capacity, his pulmonary compliance, his pulmonary resistance, his expiratory time constant, his positive end-expiratory pressure, his concentration of CO₂ in the expired air.

The ventilatory parameters for example include at least two from the following parameters: the insufflated volume, the expired volume, the tidal volume, the leak volume, the ventilatory frequency and the insufflation pressure.

Moreover, by virtue of the invention, the parameters to be modified or corrections to be made to return to an acceptable range of values are determined for the first responder, thus allowing him to act in real-time to, where needs be, modify the one or more parameters in question in the indicated way. This makes it possible to ensure that the ventilatory parameters are optimal and thus to guarantee the ventilatory assistance provided to the patient is successful, without requiring particular knowledge on the part of the first responder.

The parameters to be modified or corrections to be made may be transmitted to the user, i.e. to the first responder, by way of a display device such as a screen and/or a visual and/or audio and/or tactile indicator.

The method for ventilating the patient according to the invention allows the clinical state of the patient and his physiological characteristics to be taken into account in real-time. This makes it possible to optimally ventilate the patient depending on his clinical state during the respiratory assistance.

The ventilation device and the ventilation interface of the system for ventilating the patient used to implement the method may be such as defined above. The one or more sensors may be such as defined above. As a variant, instead of the two-way thermal mass sensor, the ventilation system may include any other type of suitable sensor of air flow rate. The microprocessor may optionally be connected by one or more wires to the one or more sensors. The microprocessor may be similar to the electronic unit such as defined above, being arranged to process the information received from the one or more sensors and the information input by the user, and to deliver information to the latter according to the method.

The invention will be better understood on reading the following detailed description, of a nonlimiting example of implementation thereof, and on examining the appended drawing, in which:

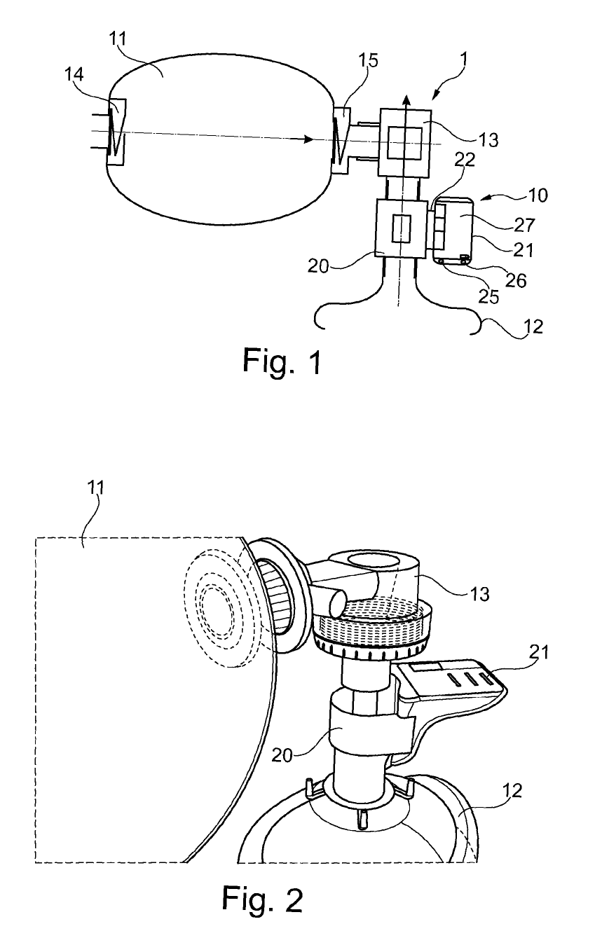

FIG. 1 is a schematic representation of a ventilation system according to the invention, incorporating a device for diagnosing the effectiveness of the ventilation of a patient according to the invention;

FIG. 2 schematically and partially shows, in perspective, the ventilation system of FIG. 1;

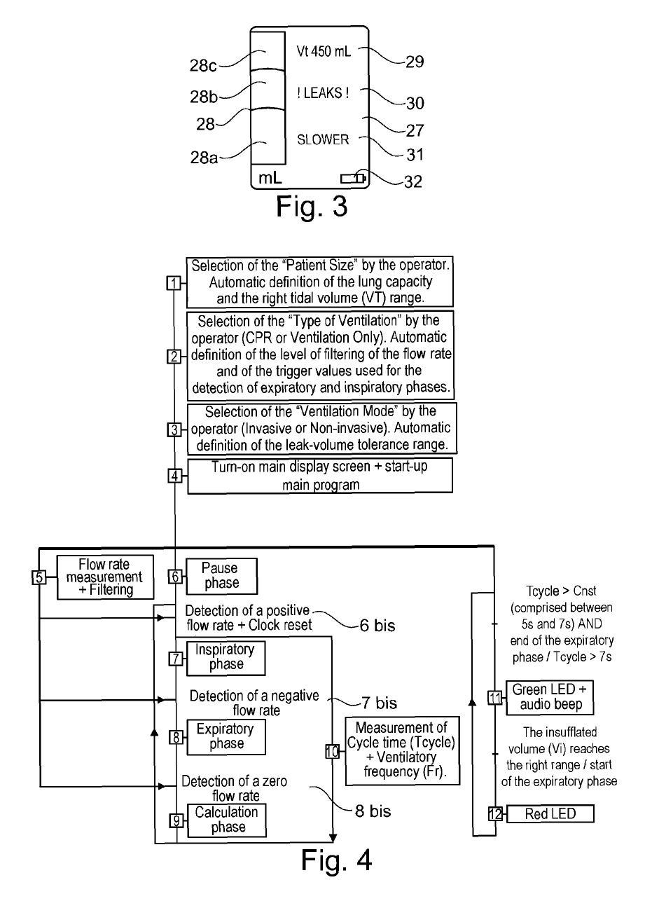

FIG. 3 schematically shows, in isolation, an example of a display of the display device of the electronic unit of the device for diagnosing the effectiveness of the ventilation of a patient of FIG. 1 or 2;

FIG. 4 schematically shows the steps of the method for ventilating a patient according to the invention; and

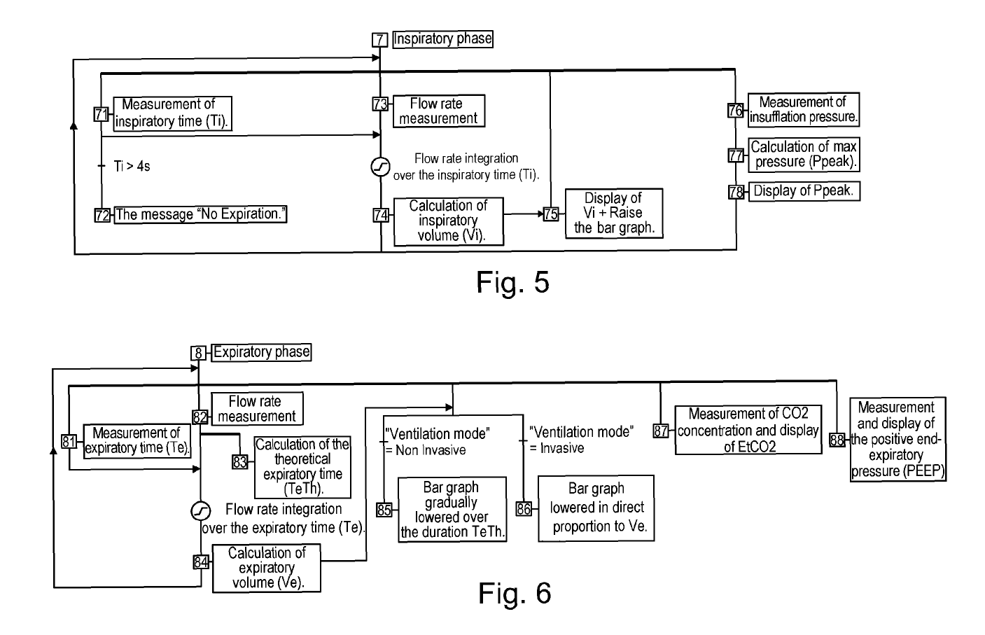

FIGS. 5 to 7 respectively detail certain steps of the method of FIG. 4.

FIG. 1 shows a ventilation system 1 for providing respiratory assistance to a patient 1 including a device 10 for analyzing the ventilatory effectiveness of the patient, which will be described below.

The ventilation system 1 includes a ventilation device 11, forming in this example a self-inflating bag. The scope of the invention is not departed from if the ventilation device is different, for example consisting of a mechanical ventilator or a flexible bag inter alia.

The ventilation system 1 may be suitable for a use such as a continuous ventilation of a patient in respiratory distress, respiratory replacement for an apneic patient, spontaneous ventilation of a patient or discontinuous ventilation of a patient in cardiac arrest or another use.

The ventilation system 1 furthermore includes a ventilation interface 12 serving to connect the ventilation system 1 to the patient, consisting in the illustrated example of a non-invasive ventilation via a mask. The mask is intended to be applied to the mouth and nose of the patient. The scope of the invention is not departed from if the ventilation interface 12 consists of an invasive ventilation via tracheal tube or any other supralaryngeal device.

The ventilation system 1 further includes a one-way expiration valve 13 placed between the ventilation device 11 and the ventilation interface 12 in order to direct air originating from the ventilation device 11 toward the ventilation interface 12 and to let the air expired by the patient escape to the atmosphere.

In this example, the ventilation device 11 is equipped with a check valve 14 that opens onto open air and that allows air to flow from the atmosphere into the ventilation device 11.

The ventilation system 1 further includes a one-way insufflation valve 15 that allows the patient to be supplied with air.

The diagnostic device 10, the ventilation device 11, the ventilation interface 12, the expiration valve 13 and the insufflation valve 15 are reversibly assembled together, for example via engagement as schematically illustrated in FIG. 1, in a way known per se.

The device 10 for diagnosing ventilatory effectiveness includes a two-way thermal mass sensor 20 able to measure in real-time air flow rates on insufflation and expiration and an electronic unit 21 connected to said sensor 20 by a disconnectable connection means 22 ensuring an electronic and mechanical connection. The two-way thermal mass sensor 20, also called a thermal mass flowmeter, may be single-use or autoclavable. It is intended to be plugged, as may be seen in FIGS. 1 and 2, on the one hand, between the insufflation valve 15 of the ventilation device 11 and the expiration valve 13, and, on the other hand, the ventilation interface 12. The sensor 20 makes it possible to measure the flow rates and volumes of air inspired and expired by measuring the specific heat capacity of the fluid, and by extension the amount of air passing therethrough in each ventilation cycle. The electronic unit 21 is configured to receive and process data relating to the air flow rates measured by the sensor 20.

In the illustrated example, the diagnostic device 10 does not include any other sensors, but it could include other sensors, for example a pressure sensor and/or a sensor of CO₂ concentration in the air, without departing from the scope of the invention.

The electronic unit 21 of the diagnostic device 10 includes a data-processing center, including a hardware portion and a software portion, a control interface or user interface comprising a display device and means for inputting data, and means for supplying electrical power such as one or more batteries. The electronic unit 21 allows ventilatory curves to be interpreted and important information relating to the effectiveness of the ventilation and various warning messages to be displayed to the first responder. If the effectiveness of the ventilation is considered to be inadequate or dangerous for the patient, the diagnostic device 10 allows the main causes of this lack of effectiveness to be identified and specific warning messages to be sent to the first responder.

The electronic unit 21 includes, in this example, as may be seen in FIG. 1, a light-emitting diode 25 or LED allowing a visual alarm to be displayed and a reset button 26, and a display device 27, shown in FIG. 3, allowing various types of warnings and messages to be displayed depending on the analysis of effectiveness performed by the electronic unit 21.

The electronic unit 21 may as a variant include or consist of a tablet computer, of a laptop, of a smartphone executing a specific application, and equipped, where needs be, with a hardware interface for interfacing with the one or more sensors and other elements of the system.

Information may be exchanged between the processing center and the one or more sensors and other elements of the system via one or more wires and/or wirelessly.

In the example illustrated in FIG. 3, the tidal volume Vt 29, which is the volume of air reaching the lungs in each respiration, expressed in ml, is displayed on the display device 27 in each ventilatory cycle. In this example, a measured tidal volume Vt of 450 ml may be read.

The inspired and expired volumes are also displayed on the screen in the form of a bar graph 28, divided into three portions in this example, forming three zones of color 28a, 28b and 28c for respectively indicating whether the volume is insufficient (28a), effective (28b) or excessive (28c) depending on the physiological characteristics of the patient.

The optimal ventilation frequencies determined by the data-processing center are transmitted to the first responder via a luminous and/or audio and/or tactile signal in order to inform him of the right rate to use. In the example of FIG. 3, a warning message 31 indicating that it is necessary to decrease the ventilation frequency appears.

In the example of FIG. 3, a warning message 30 indicating “leaks” appears, informing the first responder that it is necessary to decrease leaks, for example by repositioning the mask of the patient. Specifically, leaks are detected and calculated by measuring the discrepancy between the insufflated volume and the expired volume in each ventilatory cycle and/or observing a drop in the insufflation pressure simultaneously with an increase in flow rates.

Lastly, again in FIG. 3, a visual indicator 32 allows the level of charge of the one or more batteries to be viewed.

By virtue of this diagnostic device 10, information, delivered by the electronic unit 21, on the value of the main ventilatory parameters and on their conformity with respect to physiological and physical characteristics of the patient and the recommendations of ILCOR (the International Liaison Committee On Resuscitation) is, for each ventilation cycle, fed back to the first responder. Specifically, the measurement of the expired and insufflated volumes that is taken by virtue of the sensor 20 placed upstream of the ventilation interface 12 allows, after processing by the data-processing center of the electronic unit 21, the tidal volume, i.e. the amount of air actually being supplied to the lungs of the patient, and the leaks in each ventilation cycle to be estimated and displayed. The measurement of flow rates also allows the detection of various phases of the ventilatory cycle by virtue of specific triggers. The latter in particular allow the end of the expiration phase of the patient to be detected in order to prevent hyperventilation of the patient, which occurs when the first responder re-insufflates the patient before the end of the expiration. When the detection of the end of the expiration phase is not possible because of excessively high expiratory leaks, it may be estimated by virtue of the measurement of the expiratory time constant of the patient.

FIGS. 4 to 7 illustrate the steps of the method for ventilating a patient using the ventilation system 1, according to the invention.

With reference to FIG. 4, the method for ventilating a patient using the ventilation system 1 includes a step 1 consisting in the first responder using the user interface, in particular the inputting means, to select or indicate a physical and/or physiological characteristic of the patient to the electronic unit 21, in particular the size of the patient. The data-processing center, which receives this characteristic, is then configured to automatically define the lung capacity of the patient and the right tidal volume (V₁) range, i.e. a minimum threshold and a maximum threshold for the tidal volume.

In a step 2, the first responder may select or indicate a characteristic relating to the ventilation, in particular the type of ventilation, which is for example chosen from cardiopulmonary resuscitation (CPR) or ventilation alone. The data-processing center then automatically defines the level of filtering of the flow rate and of the trigger values used for the detection of expiratory and inspiratory phases.

In a step 3, the first responder may select another characteristic of the ventilation, for example the ventilation mode chosen from invasive or non-invasive ventilation. The data-processing center then automatically defines the leak-volume tolerance range i.e. a maximum leak-volume threshold.

In a step 4, the main screen of the display device 27 turns on and the main program of the data-processing center starts up.

In each cycle, an analysis is carried out.

In a step 5, the flow rate is measured using the sensor 20 so as to detect a pause phase 6, an inspiratory phase 7, an expiratory phase 8 and to perform a calculation phase 9. Specifically, between the pause phase 6 and inspiratory phase 7, there is a step 6bis consisting in detecting a positive flow rate generating the clock reset, this making it possible to detect that the inspiratory phase is in course. Moreover, between the inspiratory phase 7 and expiratory phase 8, in a step 7bis, a negative flow rate is detected, this making it possible to say that an expiratory phase is in course. After the expiratory phase 8, the flow rate, detected in a step 8bis, is zero, this allowing the calculation phase 9 to be triggered.

From the detection of the positive flow rate to the end of the ventilation cycle, cycle time (Tcycle) and ventilatory frequency (Fr) are measured, in a step 10.

While monitoring the ventilation cycle, and depending on the result obtained in the calculation phase 9, information is displayed and/or alarms are triggered in the form of visual and/or audio and/or tactile indicators, as will be explained below.

The detail of the method during the inspiratory phase 7 is illustrated in FIG. 5. This inspiratory phase 7 comprises the measurement of the inspiratory time Tᵢ 71. If the inspiratory time Tᵢ is longer than a preset duration, for example 4 seconds, a message 72 indicating “no expiration” is sent. It will be noted that the inspiration generally lasts between 0.5 and 2 s. Thus, if no expiration has been detected after a preset duration longer than 2 s, for example longer than 4 s after the start of the insufflation, the message 72 is displayed.

In parallel, in a step 73, the flow rate is measured, and the flow rate is integrated over the respiratory time Tᵢ, thereby allowing, in a step 74, the insufflated or inspiratory volume Vᵢ to be calculated and, in a step 75, the inspiratory volume Vᵢ to be displayed and the bar graph 28 to be raised.

In parallel, in a step 76, the insufflation pressure is measured, in a step 77, the maximum pressure Pₚₑₐₖ is measured and, in a step 78, this maximum pressure Pₚₑₐₖ is displayed.

The method in the expiratory phase 8 is detailed in FIG. 6. In the expiratory phase 8, in a step 81, the expiratory time Tₑ is measured.

In parallel, in a step 82, the flow rate is measured, and the theoretical expiratory time TeTh is calculated. The calculation of TeTh is carried out by evaluating the expiratory time constant of the patient, which is equal to 5*R*C, where R: pulmonary resistance and C: pulmonary compliance. TeTh may also be anticipated by exponential regression of the expiratory flow-rate curve. Next, the flow rate is integrated over the expiratory time Tₑ in order to deduce thereby the calculation of the expiratory volume Ve, in a step 84. When the ventilation mode is non-invasive, the bar graph 28 is gradually lowered over the duration TeTh, in a step 85. When the ventilation mode is invasive, the bar graph 28 is lowered in direct proportion to Vₑ, in a step 86.

In parallel, in a step 87, the CO₂ concentration is measured and the amount of CO₂ expired EtCO₂ displayed, for example using a measurement carried out by an optional sensor placed between the sensor 20 and the interface 12. Such a sensor is for example an NDIR (NonDispersive InfraRed) sensor allowing a measurement by infrared spectroscopy.

In parallel, in a step 88, the positive end-expiratory pressure (PEEP) is measured and displayed.

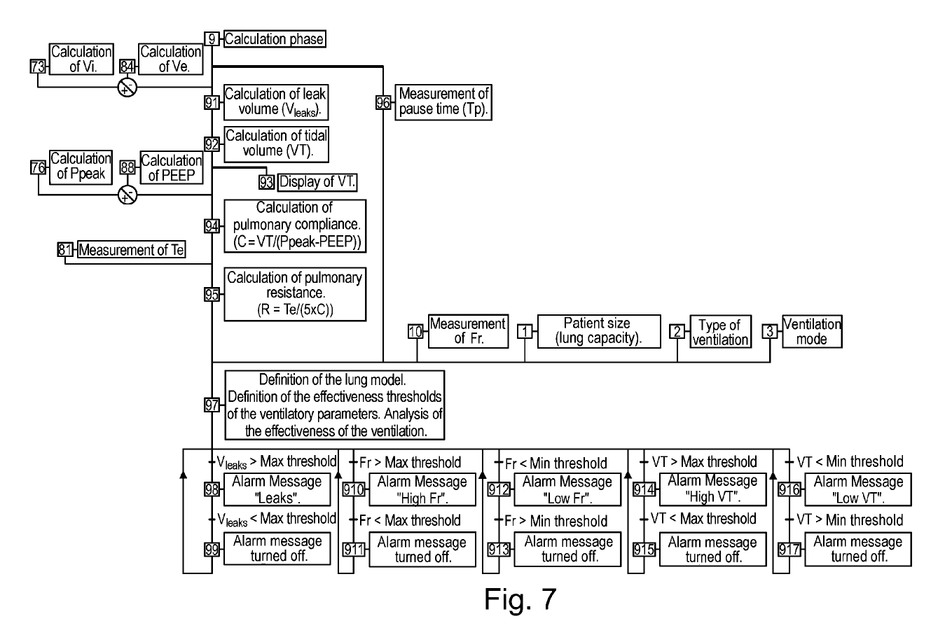

Lastly, in the calculation phase 9, as detailed in FIG. 7, the leak volume Vₗₑₐₖₛ is calculated in a step 91, then the tidal volume Vt is calculated in a step 92 and the tidal volume Vt is displayed in a step 93. In a step 94, the pulmonary compliance C is calculated using the formula C=Vₜ/(Pₚₑₐₖ−PEEP). In a step 95, the pulmonary resistance R is calculated using the formula R=Tₑ/5*C.

The pause time Tₚ is also measured in a step 96 and, using the measurement of ventilatory frequency Fr, the size of the patient, the type of ventilation and the ventilation mode and the calculations carried out in steps 94 and 95 in particular, the lung model and the effectiveness thresholds and ventilatory parameters are defined, in a step 97, and the effectiveness of the ventilation is analyzed.

If the leak volume Vₗₑₐₖₛ is higher than a maximum preset threshold, then, in a step 98, an alarm message “leaks” 30 is displayed. If the leak volume Vₗₑₐₖₛ is lower than said preset maximum threshold, in a step 99, the alarm message 30 is turned off.

In parallel, if the ventilatory frequency Fr is higher than a predefined maximum threshold, then, in a step 910, a “high ventilatory frequency” or “High Fr” alarm message is displayed, but if the ventilatory frequency is lower than the preset maximum threshold then, in a step 911, the alarm message is turned off. If the ventilatory frequency Fr is lower than a preset minimum threshold, then, in a step 912, the “low ventilatory frequency” or “low Fr” alarm message is displayed, but if the ventilatory frequency Fr is higher than said preset minimum threshold, then, in a step 913, the alarm message is turned off.

In parallel, if the tidal volume Vt is higher than a preset maximum threshold, then, in a step 914, the “high tidal volume” or “High Vt” alarm message is displayed but if the tidal volume Vt is lower than this preset maximum threshold, then, in a step 915, the alarm message is turned off. If the tidal volume Vt is lower than a preset minimum threshold, then, in a step 916, the “low tidal volume” or “low Vt” alarm message is displayed. When the tidal volume Vt is higher than a preset minimum threshold then, in a step 917, the alarm message is turned off.

In a step 11 illustrated in FIG. 4, a light-emitting diode 25 of green color is turned on and an audio signal is emitted when the cycle time is longer then a constant comprised in a preset range of values, for example between 5 and 7 seconds, and the end of the expiratory phase is detected or the cycle time exceeds a preset threshold value, for example 7 seconds. This luminous and audio signal makes it possible to indicate to the first responder the right time for the insufflation. When the insufflated volume Vi reaches the right range or the start of the expiratory phase is detected, then, in a step 12, the visual indicator such as a light-emitting diode 25 of red color is turned on to warn the first responder. The right range of the insufflated volume Vi is determined in steps 1 and 97. The volume Vi is right if there are no leaks. Otherwise, the right volume is corrected depending on the leaks. The optimal cycle time is based on the pulmonary characteristics of the patient, such as his pulmonary compliance and pulmonary resistance.

The leak volume may also be expressed in percent of the insufflated volume and have a preset maximum threshold, for example comprised between about 20% and 40% of the insufflated volume. The maximum threshold of the respiratory frequency Fr is for example comprised between about 12 and 20 cycles per minute and the minimum threshold of the ventilatory frequency Fr is for example comprised between about 8 and 12 cycles per minute. As for the tidal volume Vt, the preset maximum threshold is for example comprised between about 500 ml and 700 ml and the preset minimum threshold is for example comprised between about 300 ml and 500 ml.

By virtue of the invention, the first responder may immediately have access to information on the leak volume, the ventilatory frequency Fr, the tidal volume Vt and very rapidly influence the one or more parameters to be corrected, where needs be, in order to re-establish an optimal ventilation for the patient. The iteration of the steps of the method in each ventilation cycle of the patient allows the first responder to continuously adapt to the evolution of the clinical state of the patient and to modulate the parameters indicated on the display device 27, without having in-depth knowledge of the ventilation system or respiratory physiology.

The invention is of course not limited to the example just described.

In particular, the system may be adapted to a pediatric or neonatal use and the thresholds described above may change accordingly.

Throughout the description, the expression “including a” must be understood as being synonymous with the expression “comprising at least one”.

Ranges of values are understood to be inclusive of limits unless otherwise specified.

1: A device for diagnosing the ventilatory effectiveness of a patient under respiratory assistance, intended to interact with a system for ventilating the patient, the device including:
a two-way thermal mass sensor able to measure in real-time air flow rates on insufflation and on expiration;
an electronic unit connected to said sensor, configured to receive and process data relating to the air flow rates measured by the sensor, the electronic unit including:
a user interface comprising a display device and means for inputting data;
a data-processing center, the data-processing center operating according to algorithms programmed to acquire, to process and to display data, to analyze the effectiveness of the ventilation in real-time and to manage alarms; and
means for supplying electrical power.

2: The device as claimed in claim 1, further comprising a disconnectable connection between the sensor and the electronic unit.

3: The device as claimed in claim 1, wherein the display device is a screen and the means for supplying electrical power being a battery.

4: The device as claimed in claim 1, wherein the sensor is single-use.

5: The device as claimed in claim 1, further comprising at least one other sensor, chosen from the following sensors:
a pressure sensor, and
a sensor of CO₂ concentration in the air.

6: The device as claimed in claim 1, wherein the means for inputting data is configured to allow physical and/or physiological characteristics of the patient to be input into the electronic unit, and/or characteristics relating to the ventilation, including the type of ventilation, to the type of ventilation device and/or the type of ventilation interface to be input.

7: The device as claimed in claim 6, wherein the physical and/or physiological characteristics of the patient or physiological parameters of the patient measured by the sensor comprises at least two from the following characteristics or parameters: the size of the patient, his lung capacity, his pulmonary compliance, his pulmonary resistance, his expiratory time constant, his positive end-expiratory pressure, his concentration of CO₂ in the expired air.

8: The device as claimed in claim 7, wherein the data-processing center is configured to, throughout the duration of the ventilatory assistance provided to the patient, in particular in each ventilation cycle, analyze said characteristics and the physiological parameters measured, in particular in each ventilation cycle, by the sensor, in order to deduce therefrom ideal ventilatory parameters for an optimal ventilation of said patient, and for each ventilatory parameter, a minimum and/or maximum threshold.

9: The device as claimed in claim 8, wherein the ventilatory parameters include at least two from the following parameters: the insufflated volume, the expired volume, the tidal volume, the leak volume, the ventilatory frequency and the insufflation pressure.

10: The device as claimed in claim 8, wherein the data-processing center is configured to receive ventilatory parameters measured by the sensor and to compare them to said thresholds, throughout the duration of the ventilatory assistance provided to the patient, in each ventilation cycle.

11: The device as claimed in claim 10, wherein the data-processing center is configured to, throughout the duration of the ventilatory assistance provided to the patient, in each ventilation cycle, and for each ventilatory parameter, in case of value of a measured ventilatory parameter higher than a corresponding maximum threshold and/or lower than a corresponding minimum threshold, generate an alarm and/or a piece of information on the one or more ventilatory parameters to be modified or corrections to be carried out to achieve an optimal ventilation.

12: The device as claimed in the claim 11, wherein the electronic unit is configured to transmit to the user via the display device the parameters to be modified or corrections and/or the electronic unit includes a visual and/or audio and/or tactile indicator and is configured to transmit to the user via said indicator the parameters to be modified or corrections.

13: A ventilation system for providing respiratory assistance to a patient, including a device for diagnosing the effectiveness of the ventilation of the patient as claimed in claim 1, and a ventilation device chosen from the group consisting of: a flexible bag, a self-inflating bag and a mechanical ventilator.

14: The ventilation system as claimed in claim 13, including a ventilation interface chosen from the group consisting of: an invasive ventilation via tracheotomy or tracheal tube, and a non-invasive ventilation via a mask, the two-way thermal mass sensor being located between the ventilation device and the ventilation interface.

15: A method for determining the ventilatory effectiveness of a patient using a ventilation system for providing respiratory assistance to a patient, including a device for diagnosing the effectiveness of the ventilation of the patient under respiratory assistance, intended to interact with a system for ventilating the patient, the device including a two-way thermal mass sensor able to measure in real-time air flow rates on insufflation and on expiration, an electronic unit connected to said sensor, configured to receive and process data relating to the air flow rates measured by the sensor, the electronic unit including a user interface comprising a display device and means for inputting data, a data-processing center, the data-processing center operating according to algorithms programmed to acquire, to process and to display data, to analyze the effectiveness of the ventilation in real-time and to manage alarms, and means for supplying electrical power, and a ventilation device chosen from the group consisting of: a flexible bag, a self-inflating bag and a mechanical ventilator, comprising:
a) allowing physical and/or physiological characteristics of the patient to be input into the electronic unit, and/or characteristics relating to the ventilation, in particular relating to the type of ventilation, to the type of ventilation device and/or to the type of ventilation interface to be input;
b) measuring the physiological parameters of the patient using the sensor;
c) analyzing the characteristics input in step a) and the parameters measured in step b);
d) deducing therefrom, in real-time, ideal ventilatory parameters for an optimal ventilation of said patient, and for each ventilatory parameter, a minimum and/or maximum threshold;
e) measuring in real-time the ventilatory parameters of the patient;
f) comparing the measured ventilatory parameters to said thresholds, respectively;
g) for each ventilatory parameter, in case of value of a measured ventilatory parameter higher than a corresponding maximum threshold and/or lower than a corresponding minimum threshold, generating an alarm and/or a piece of information on the one or more parameters to be modified or corrections to be carried out to achieve an optimal ventilation;
h) repeating steps b) to g) throughout the duration of the ventilatory assistance provided to the patient, in each ventilation cycle.

16: The method as claimed in claim 15, wherein the physical and/or physiological characteristics of the patient comprise at least two from the following characteristics or parameters: the size of the patient, his lung capacity, his pulmonary compliance, his pulmonary resistance, his expiratory time constant, his positive end-expiratory pressure, his concentration of CO₂ in the expired air.

17: The method as claimed in claim 15, wherein the ventilatory parameters include at least two from the following parameters: the insufflated volume, the expired volume, the tidal volume, the leak volume, the ventilatory frequency and the insufflation pressure.

18: The method as claimed in claim 15, wherein the parameters to be modified or corrections are transmitted to the user by way of a display device including a screen and/or a visual and/or audio and/or tactile indicator.
